(19) United States
(12) Patent Application Publication
(10) Pub. No.: US 2021/0038140 A1
(43) Pub. Date: Feb. 11, 2021

(54) ASSESSMENT SYSTEM AND ASSESSMENT METHOD

(71) Applicant: KANEKA CORPORATION, Osaka-shi, Osaka (JP)

(72) Inventor: Yoshiyuki NASUNO, Osaka-shi (JP)

(21) Appl. No.: 16/977,933

(22) PCT Filed: Mar. 11, 2019
(86) PCT No.: PCT/JP2019/009645

§ 371 Date: Sep. 3, 2020

(30) Foreign Application Priority Data
Mar. 13, 2018 (JP) 2018-045208

(51) Int. Cl. A61B 5/16; A61B 5/00; A61B 5/0402; G06Q 30/02

(52) U.S. Cl. CPC A61B 5/163; A61B 5/4064; A61B 5/7296; A61B 2560/0242; A61B 5/7264; A61B 5/74; G06Q 30/02; A61B 5/0402

(57) ABSTRACT

The present invention conducts an analysis on a user who has actually seen a commercial product or the like. An assessment system is provided with a biological information measurement device, a response state assessment unit, and a notification unit. The biological information measurement device measures biological information of a customer who has been presented with a provided commercial product and/or a provided service. The response state assessment unit identifies a response state of the customer who has been provided with the presentation, by making an assessment based on the biological information measured by the biological information measurement device. The notification unit gives notice based on the customer's response state identified by the response state assessment unit.

### TECHNICAL FIELD

The present invention relates to an assessment system and an assessment method.

### BACKGROUND ART

In recent years, measurement of biological information of users with biosensors is widely performed. Measured biological information is used not only in the medical field but also in various fields.

An example of a technique related to use of such biological information is described in Patent Document 1. With the technique described in Patent Document 1, the user's biological information is measured with a biosensor, such as a brain wave sensor. The condition, such as the degree of fatigue and stress, of the user is identified in accordance with the measured biological information. Information on commercial products suitable for the identified condition is displayed for the user as information on recommended commercial products.

### PATENT DOCUMENT 1

Japanese Unexamined Patent Application, Publication No. 2001-344352

### DISCLOSURE OF THE INVENTION

### Problems to be Solved by the Invention

By using an existing technique, such as the technique described in Patent Document 1, a commercial product and/or a service suitable for the condition, such as the degree of fatigue and stress, of a user at that time can be newly proposed to the user. The existing technique, such as the technique described in Patent Document 1, just newly proposes a commercial product or the like to the user and does not conduct an analysis on a user who has actually encountered a commercial product or the like.

The present invention is made in view of such a situation. It is an object of the present invention to provide an assessment system and an assessment method for conducting an analysis on a user who has actually encountered a commercial product or the like.

### Means for Solving the Problems

An assessment system according to the present invention includes a measurement unit that measures biological information of a customer who has been presented with at least any one of a provided commercial product and a provided service, an assessment unit that identifies a response state of the customer provided with the presentation, by making an assessment based on the biological information measured by the measurement unit, and a notification unit that gives notice based on the response state of the customer, identified by the assessment unit.

An assessment method according to the present invention is an assessment method that is executed by an assessment system, and includes a measurement process in which a measurement unit measures biological information of a customer who has been presented with at least any one of a provided commercial product and a provided service, an assessment process in which an assessment unit identifies a response state of the customer provided with the presentation, by making an assessment based on the biological information measured by the measurement unit, and a notification process in which a notification unit gives notice based on the response state of the customer, identified by the assessment unit.

### Effects of the Invention

According to the present invention, it is possible to conduct an analysis on a user who has actually encountered a commercial product or the like.

### BRIEF DESCRIPTION OF THE DRAWINGS

FIG. 1 is a block diagram showing an example of the overall configuration of an assessment system according to an embodiment of the present invention.

FIG. 2 is a block diagram showing an example of the configuration of an assessment device according to the embodiment of the present invention.

FIG. 3 is a table showing an example of the data structure of an assessment information database, which is constructed by the assessment device according to the embodiment of the present invention.

FIG. 4 is a flowchart illustrating the flow of a response state assessment process that is executed by an information collection device according to the embodiment of the present invention.

### PREFERRED MODE FOR CARRYING OUT THE INVENTION

Hereinafter, an example of embodiments of the present invention will be described with reference to the attached drawings.

### System Configuration

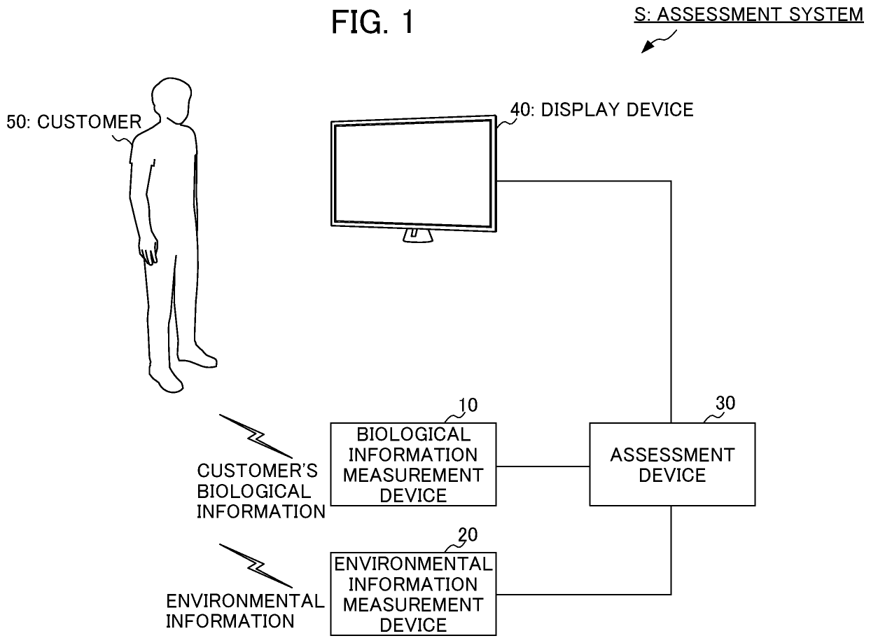

FIG. 1 is a block diagram showing the overall configuration of an assessment system S according to the present embodiment. As shown in FIG. 1, the assessment system S includes a biological information measurement device 10, an environmental information measurement device 20, an assessment device 30, and a display device 40. FIG. 1 shows a customer 50 that is an object on which the assessment system S executes a process.

These devices included in the assessment system S are connected to one another via a network so as to communicate with one another. Communication between the devices may be performed in conformity with any communication mode, and the communication mode is not limited. The communication connection may be a wireless connection or may be a wired connection. Communication between the devices may be directly performed by the devices without an intervening network.

The assessment system S is installed in, for example, a place where the customer 50 is presented with a provided commercial product, which is provided by a business operator, or a provided service, which is provided by a business operator. This presentation may be provided by presenting a provided commercial product and a provided service themselves or may be provided by presenting information on a provided commercial product and a provided service.

The assessment system S is installed in, for example, exhibition halls where provided commercial products can be actually seen or touched, experience facilities where provided services presented can be actually experienced, or the like. Examples of provided commercial products that are displayed in exhibition halls include model houses, model rooms, cars, domestic appliances, art, and jewelry. Examples of provided services that can be actually experienced in experience facilities include haircut or hairdressing services, provided services of food and drink, and provided services of entertainment. Hereinafter, description will be made on the assumption that the assessment system S is installed in a model house. In this case, the customer 50 is a visitor who becomes interested in the model house and visits the model house.

The assessment system S thus configured measures biological information of the customer 50 who has been presented with at least any one of a provided commercial product and a provided service. The assessment system S identifies a response state of the customer 50 who has been provided with the presentation, by making an assessment based on the measured biological information. The assessment system S gives notice based on the identified response state of the customer 50. Therefore, with the assessment system S, it is possible to conduct an analysis on a user who has actually encountered a commercial product or the like. The assessment system S can be used as a sales promotion tool that, for example, provides information that enables a response state, which is subjective information of the customer 50, to be studied from an objective viewpoint and that makes it possible to evaluate commercial products.

Next, the devices included in the assessment system S will be described in detail. The biological information measurement device 10 includes a function of measuring biological information of the customer 50 (hereinafter, referred to as customer's biological information). The biological information measurement device 10 measures the biological information of the customer 50 by using, for example, any one or any combination of a brain wave sensor, an electrocardiographic sensor, and a Doppler sensor.

A brain wave sensor has a higher response speed than the other sensors, so the brain wave sensor has such a feature suitable for measuring an instantaneous change. In contrast, the other sensors take a data collection time of about 30 seconds to one minute. A Doppler sensor has such a feature that the Doppler sensor is capable of measuring information on a heart rate, a rate of respiration, and body movements with no contact with the body of the customer 50. A Doppler sensor is capable of measuring, for example, a rate of respiration, a ratio between an inspiration time and an expiration time, a depth of movement of a chest at the time of respiration, or the like. In contrast, the other sensors need to contact the body of the customer 50 for measurement. An electrocardiographic sensor has, for example, such a feature that the electrocardiographic sensor is capable of measuring heart rate variability more accurately than a Doppler sensor.

A biosensor that is used in the biological information measurement device 10 is determined according to the features of these biosensors and a type of biological information to be measured. Hereinafter, it is assumed that the biological information measurement device 10 measures the customer's biological information by using two-type biosensors. For example, the biological information measurement device 10 measures a variation in brain waves by using a headphone-type brain wave sensor that is electrically in contact with the body at two points, that is, the forehead and ear lobe of the customer 50. The biological information measurement device 10 also measures a variation in heartbeat, which is one piece of the customer's biological information, by using a two-point contact-type electrocardiographic sensor of which each one electrode contacts with one of both thumbs of the customer 50. The biological information measurement device 10 generates the customer's biological information by associating the variation in brain waves and the variation in heartbeat, measured by these biosensors, with measurement time in time series. The biological information measurement device 10 transmits the generated customer's biological information to the assessment device 30.

The environmental information measurement device 20 includes a function of measuring environmental information indicating the characteristics of an environment around the customer 50. The environmental information measurement device 20 measures environmental information by using, for example, any one or any combination of a temperature sensor, a humidity sensor, an air quality sensor, a particle counter, an illuminance sensor, an atmospheric pressure sensor, and an acceleration sensor. In this case, examples of environmental information include physical quantities, such as temperature, humidity, air quality (for example, the concentration of carbon dioxide and the amount of fine particles in air), illuminance, atmospheric pressure, and acceleration. It may be better to measure an acceleration when, for example, the environment around the customer 50 is inside a mobile unit, such as an automobile.

Environmental information may contain not only such physical quantities but also other information. Environmental information may contain, for example, information indicating any one of a place where a mind is generally easy to be uplifted and a place where a mind is generally easy to be settled as an environment around the customer 50. In more details, environmental information may contain information indicating any one of places, such as a scenic area, a noted place and historic scene, a resort, and a so-called power sport, which are generally places where a mind is uplifted, as other information. Similarly, environmental information may contain information indicating any one of places, such as home, an accommodation, and a coffee shop, which are generally places where a mind is easy to be settled, as other information. Environmental information may contain, for example, information, such as an operation mode and a set temperature of an air conditioner, as an environment in a model house, as other information.

For example, an administrator of the assessment system S inputs information other than these physical quantities to the environmental information measurement device 20 or the assessment device 30. Alternatively, the environmental information measurement device 20 or the assessment device 30 acquires information indicating a place by, for example, comparing positional information measured in compliance with a GPS (Global Positioning System) or the like with map information.

The environmental information measurement device 20 generates environmental information by associating the physical quantities acquired through measurement with the sensors, other information indicating a place, and the like with measurement time. The environmental information measurement device 20 transmits the generated environmental information to the assessment device 30.

The assessment device 30 includes a function of identifying a response state of the customer 50 who has been presented with a provided commercial product or the like, by making an assessment based on the customer's biological information acquired from the biological information measurement device 10 and the environmental information acquired from the environmental information measurement device 20. The assessment device 30 includes a function of executing a process for giving notice based on the identified response state of the customer 50. A process for giving notice is implemented by, for example, transmitting notification information to the display device 40.

The display device 40 is a display device, such as a liquid crystal display connected to the assessment device 30 and a display unit in a tablet terminal. The display device 40 displays notification information received from the assessment device 30.

The customer 50 is able to study a response state, which is subjective information of the customer 50, from an objective viewpoint by consulting the notification information. Therefore, the customer 50 is able to evaluate a provided commercial product or the like in accordance with an index that is a customer's own response state. The display device 40 may give notice to a business operator that sells a provided commercial product or the like. In this case, a business operator is able to obtain a response state of the customer 50 as an index of a response and reaction of the customer 50 to a provided commercial product or the like. Thus, the assessment system S functions as a beneficial sales promotion tool for a business operator that sells a provided commercial product or the like.

The devices included in the assessment system S are described above. In the drawing, one of each device is shown; however, this is only illustrative. The assessment system S may include any number of each. For example, multiple sets of the biological information measurement device 10, the environmental information measurement device 20, and the display device 40 may be provided in association with multiple customers 50. For example, the single assessment device 30 may be configured to collective execute an assessment process on these multiple sets.

### Configuration of Assessment Device

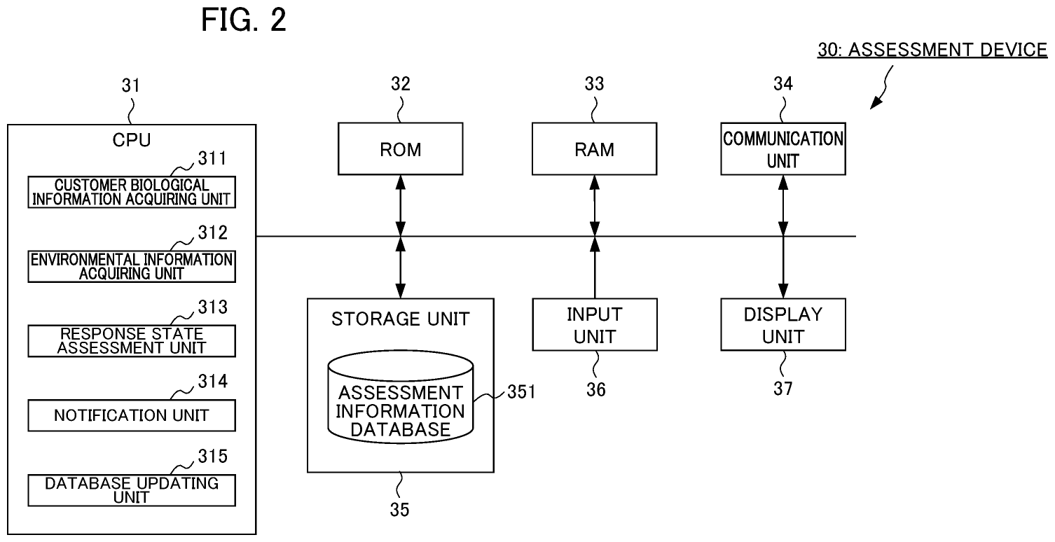

Next, the configuration of the assessment device 30 will be described with reference to the block diagram of FIG. 2. As shown in FIG. 2, the assessment device 30 includes a CPU (central processing unit) 31, ROM (read-only memory) 32, RAM (random access memory) 33, a communication unit 34, a storage unit 35, an input unit 36, and a display unit 37. These units are bus-connected by a signal line and transmit or receive signals to or from one another.

The CPU 31 executes various processes in accordance with a program stored in the ROM 32 or a program loaded from the storage unit 35 onto the RAM 33. Data and the like that are used by the CPU 31 to execute various processes are also stored as needed on the RAM 33.

The communication unit 34 executes communication control for the CPU 31 to communicate with the other devices included in the assessment system S. The storage unit 35 comprises semiconductor memory, such as DRAM (dynamic random access memory), and stores various data.

The input unit 36 comprises various buttons and a touch panel, or an external input device, such as a mouse and a keyboard, and inputs various pieces of information in response to a user's instruction operation. The display unit 37 comprises a liquid crystal display or the like and displays an image corresponding to image data output by the CPU 31.

In the assessment device 30, these units cooperate to execute a response state assessment process. The response state assessment process is a series of processes in which the assessment device 30 identifies a response state of the customer 50 who has been presented with a provided commercial product or the like, by making an assessment based on the customer's biological information or the like and gives notice about the identified response state.

When the response state assessment process is executed, a customer biological information acquiring unit 311, an environmental information acquiring unit 312, a response state assessment unit 313, a notification unit 314, and a database updating unit 315 function on the CPU 31 as shown in FIG. 2. An assessment information database 351 is also set in one area of the storage unit 35.

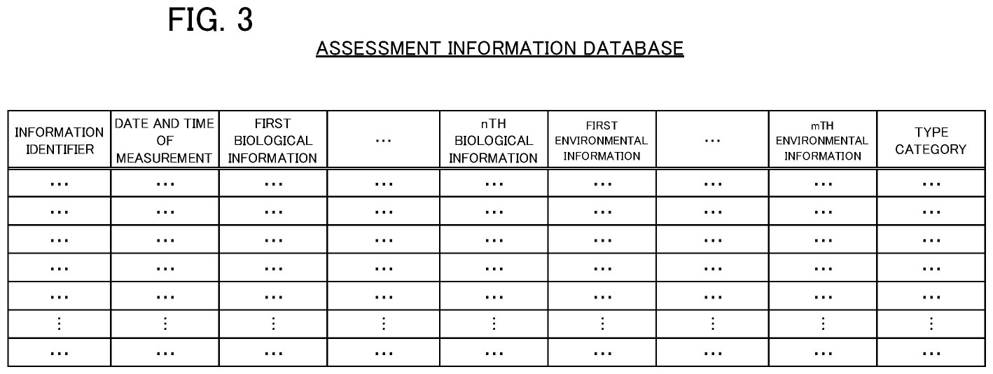

The assessment information database 351 is a database that stores various pieces of information that the assessment device 30 uses to execute the response state assessment process. An example of the specific data structure of the assessment information database 351 will be described with reference to FIG. 3.

As shown in FIG. 3, in the assessment information database 351, multiple pieces of biological information assigned with an identifier are stored as one record respectively in association with pieces of other information, such as associated pieces of environmental information. For example, pieces of biological information and associated pieces of other information, measured in the single response state assessment process by the biological information measurement device 10, are stored as one record. These records are, for example, pieces of information acquired by measuring the customer 50 through the response state assessment process in the past.

Not limited to this configuration, to improve the accuracy of assessment in the response state assessment process by providing enough records stored in the database, it may be better to acquire information, such as biological information, from persons other than the customer 50. Specifically, to acquire various data, it may be better to acquire biological information by measuring various persons in various places. It may be better to acquire biological information by performing a measurement in, for example, the above-described place where a mind is generally easy to be uplifted and the above-described place where a mind is generally easy to be settled. For persons to be measured, it may be better to acquire biological information from various persons whatever their age or sex.

For the same person, biological information in various states may be acquired. A data set of biological information may be generated by acquiring biological information in various states for the same person, for example, in a relaxed state in a living room, in a sleeping state, in a studying or working state, in an excited state of watching a sport event, a state of admiring scenery in a scenic area, and a state of intentionally taking a stress test. Furthermore, such data sets may be generated for a large number of persons.

In this way, when a measurement is performed for various persons other than the customer 50 in various places, a measurement may be performed by the biological information measurement device 10 and the environmental information measurement device 20 included in the assessment system S or may be performed by other measurement devices.

Each of the records contains, for example, an information identifier, date and time of measurement, first biological information to nth biological information (n is the value of any natural number), first environmental information to mth environmental information (m is the value of any natural number), and a type category as columns. Specific details of information corresponding to each of these columns will be described.

An information identifier is an identifier for identifying each record. It may be enough that an information identifier is information unique to each of the records, and an information identifier may be any information. It may be better to use, for example, a number indicating the order in which the record is stored in the assessment information database 351 or an ID (identifier) that is assigned in accordance with a predetermined rule, as an information identifier.

Date and time of measurement is information indicating date and time at which pieces of biological information and the like contained in the record are measured. Date and time of measurement is, for example, time information from a start of measurement of biological information and the like to an end of measurement of biological information and the like.

First biological information to nth biological information are pieces of biological information, measured by the biological information measurement device 10 and the like, and are respectively contained in different columns for types of biological information. For example, first biological information is brain wave, second biological information is heart rate, third biological information is rate of respiration, . . . , and nth biological information is depth of respiration.

First environmental information to mth environmental information are pieces of environmental information, measured by the environmental information measurement device 20 and the like, and are respectively contained in different columns for types of environmental information. For example, first environmental information is temperature, second environmental information is humidity, third environmental information is air quality, . . . and mth environmental information is illuminance.

A type category is information indicating a result obtained by classifying a person of which biological information is to be acquired in accordance with predetermined criteria. Classification may be performed in accordance with, for example, attributes, such as age, sex, and occupation, of a person of which the biological information is to be acquired. There are individual differences in the degree of influence of biological information on a response state. Therefore, classification is performed in accordance with information on the attributes and the like of persons, which are estimated to correlate with individual differences. The response state assessment unit 313 (described later) is capable of further highly accurately making an assessment by assessing biological information by using the type category. The response state assessment unit 313 is capable of further highly accurately making an assessment by, for example, assessing biological information through a comparison between pieces of biological information of the same type. These pieces of information in the assessment information database 351 are updated as needed by the database updating unit 315 (described later).

The customer biological information acquiring unit 311 acquires the customer's biological information by receiving the customer's biological information from the biological information measurement device 10. The customer biological information acquiring unit 311 outputs the acquired customer's biological information to the response state assessment unit 313 (described later) and the database updating unit 315 (described later).

The environmental information acquiring unit 312 acquires environmental information by receiving the environmental information from the environmental information measurement device 20. The environmental information acquiring unit 312 outputs the acquired environmental information to the response state assessment unit 313 (described later) and the database updating unit 315 (described later).

The response state assessment unit 313 identifies the response state of the customer 50 by making an assessment based on at least one customer's biological information input from the customer biological information acquiring unit 311. As a precondition for describing an assessment that is made by the response state assessment unit 313, a procedure leading to an assessment will be described. In the present embodiment, the customer 50 who visits a model house wears a biosensor outside the model house. The biological information measurement device 10 immediately starts performing a measurement for the customer 50. The customer's biological information acquired as a result of the measurement is acquired by the customer biological information acquiring unit 311 as described above and output to the response state assessment unit 313.

In this state, a business operator that sells houses by taking advantage of the model house explains the outline of the model house to the customer 50 before the customer 50 enters the model house. After the explanation, the customer 50 enters the model house and experiences the indoor environment of the model house. As a result, the response state assessment unit 313 makes an assessment based on the customer's biological information of the customer 50 who is experiencing the indoor environment of the model house and identifies the response state of the customer 50.

Examples of the response state include various states such as comfort, affection, emotions, a burden on the body, and mood. The present embodiment focuses on comfort depending on an environment. A high-comfort state is defined as a state where many α waves appear in brain waves and parasympathetic nerves are predominant.

α waves are identified by, for example, separating frequency components in a middle frequency band resulting from frequency-resolved brain waves obtained through Fourier transformation as the customer's biological information. For parasympathetic nerves, when frequency components in a high frequency band (for example, 0.20 Hz to 0.35 Hz) resulting from a power spectrum analysis on frequency components of periodic variations in heart rate are more than the other frequency components, it may be determined that parasympathetic nerves are predominant.

As an example of an assessment method based on these pieces of information using the response state assessment unit 313, three first to third assessment methods will be described. The first assessment method is a method that makes an assessment in accordance with the intensity of α waves. Specifically, in the first assessment method, a threshold for α waves is set. When the absolute intensity of α waves exceeds the threshold, comfort is assessed as a high state. When multiple thresholds are set, comfort is assessed in levels.

Comfort may be assessed in accordance with a relative rate of change in the intensity of α waves. In this case, a relative rate of change in the intensity of α waves before and after entering a model house is defined as {A0×(Intensity of α waves after entry)}/(Intensity of α waves before entry). Here, A0 is a predetermined constant. By providing a threshold for the relative rate of change in the intensity of α waves, comfort is also assessed in accordance with a relative rate of change as in the case of making an assessment in accordance with the absolute intensity. In the first assessment method, an assessment may be made from information close to raw data, so it is advantageous in that objectivity is high, and it is possible to reduce the possibility of leading to misunderstanding at the time of providing an explanation to the customer.

The second assessment method is a method that also takes the degree of predominance of parasympathetic nerves into consideration in addition to the first assessment method. In the second assessment method, comfort is defined as (Comfort)=A1×(Intensity of α waves)+A2×(Degree of predominance of parasympathetic nerves). Here, A1 and A2 are predetermined constants. A threshold for the comfort is set. When comfort exceeds the threshold, comfort is assessed as a high state. When multiple thresholds are set, comfort is assessed in levels. Not only such a linear expression is used to define comfort but also a quadratic or higher-order expression may be used to define comfort.

As in the case of the first assessment method, comfort may be assessed in accordance with a relative rate of change in the intensity of α waves and the degree of predominance of parasympathetic nerves. In this case, in the above-described definition (Comfort)=A1×(Intensity of α waves)+A2×(Degree of predominance of parasympathetic nerves), (Intensity of α waves) is replaced with (Relative rate of change in intensity of α waves), and (Degree of predominance of parasympathetic nerves) is replaced with (Relative rate of change in degree of predominance of parasympathetic nerves)={A3×(Degree of predominance of parasympathetic nerves after entry)}/(Degree of predominance of parasympathetic nerves before entry). By providing a threshold of the intensity of α waves for the relative rate of change and the degree of predominance of parasympathetic nerves for the relative rate of change, comfort is also assessed in accordance with a relative rate of change as in the case of making an assessment in accordance with the absolute intensity. The second assessment method also takes the degree of predominance of parasympathetic nerves into consideration in addition to α waves. Therefore, in comparison with the first assessment method, the accuracy of assessing comfort further improves.

The third assessment method is a method that further uses a type category in the first assessment method and the second assessment method. In the third assessment method, the above-described values of A0, A1, A2, and A3 are varied for each type category. Thus, an assessment is made according to the tendency of each type, so it is possible to further highly accurately make an assessment.

The response state assessment unit 313 identifies the response state of the customer 50 by making an assessment by the assessment method as described above. The response state assessment unit 313 outputs the identified response state to the notification unit 314. In the present embodiment, the response state assessment unit 313 outputs comfort identified as a result of assessment to the notification unit 314. The notification unit 314 generates notification information in accordance with the response state input from the response state assessment unit 313. Notification information may be, for example, information that indicates a response state itself identified by the response state assessment unit 313.

Information other than a response state may be contained in notification information. For example, by comparing the biological information of the customer 50 with the biological information of each of the records stored in the assessment information database 351, information indicating that the biological information is approximate to biological information under another environment may be contained in notification information. For example, a message, such as “Same state as in the case of being in a scenic area” and “Same as in the case of being relaxed in the living room”, may be generated and contained in notification information.

The notification unit 314 transmits the generated notification information to the display device 40 to give notice of the generated notification information to the customer 50 or the like. As described above, the display device 40 having received notification information displays the received notification information. Notice may be given with a method other than displaying notice on the display device 40. Notice may be given by, for example, outputting speech from a speaker using notification information as speech data.

The database updating unit 315 stores the customer's biological information input from the customer biological information acquiring unit 311 and the environmental information input from the environmental information acquiring unit 312 in the associated fields of the assessment information database 351. As described above in the description of the assessment information database 351, the database updating unit 315 also stores biological information and the like obtained by measuring various persons in various places in the associated fields of the assessment information database 351.

Furthermore, the database updating unit 315 makes a type classification of biological information and the like to be newly stored and stores information indicating the result of the type classification in the field “type category” of the assessment information database 351. A type classification may be made by comparing new biological information and the like with existing biological information and the like stored in the assessment information database 351. In accordance with a comparison result, approximate pieces of biological information may be, for example, classified as the same type. Other than that, a type classification may be made in accordance with, for example, the attributes, such as age, sex, and occupation, of a person of which the biological information is to be acquired, as described above. Alternatively, a type classification may be made by classifying a person who exhibits a similar response for a change in state by using AI (artificial intelligence).

### Response State Assessment Process

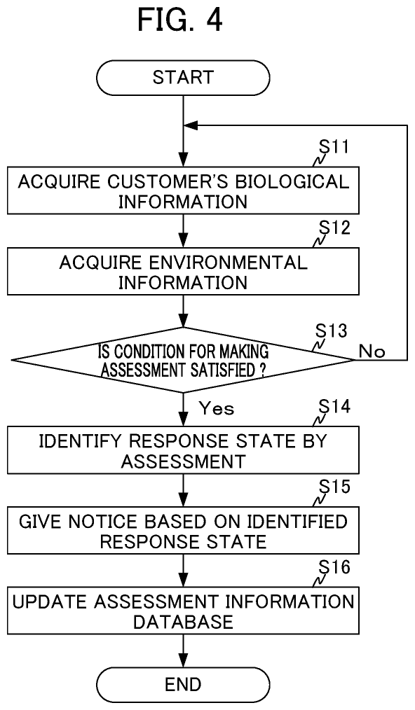

Next, the flow of a response state assessment process that is executed by the assessment device 30 will be described with reference to the flowchart of FIG. 4.

In step S11, the customer biological information acquiring unit 311 acquires the customer's biological information by receiving the customer's biological information from the biological information measurement device 10. The customer biological information acquiring unit 311 outputs the acquired customer's biological information to the response state assessment unit 313 (described later) and the database updating unit 315 (described later).

In step S12, the environmental information acquiring unit 312 acquires environmental information by receiving the environmental information from the environmental information measurement device 20. The environmental information acquiring unit 312 outputs the acquired environmental information to the response state assessment unit 313 (described later) and the database updating unit 315 (described later). For the sake of convenience of illustration, the description is made such that step S12 is executed after step S11 is executed; however, step S11 may be executed after step S12 is executed or step S11 and step S12 may be executed in parallel.

In step S13, the response state assessment unit 313 determines whether a condition for making an assessment is satisfied. A condition for making an assessment is, for example, a condition in which a predetermined period of time has elapsed from when a measurement of the customer's biological information and environmental information is started. In the case of the present embodiment, a condition in which the customer 50 enters a model house and has experienced the indoor environment of the model house for a predetermined period of time may be used as a condition for making an assessment. In this case, it may be better for, for example, a business operator that sells houses by taking advantage of a model house to input by using the input unit 36 a situation in which a condition for making an assessment is satisfied. Alternatively, for example, a motion detector may be installed at the entrance of a model house, and whether a condition for making an assessment is satisfied may be determined in accordance with detection of the customer 50 with the motion detector. Alternatively, for example, without providing a condition for making an assessment, an assessment may be made immediately after a measurement of the customer's biological information and environmental information is started.

When such a condition for making an assessment is not satisfied, a negative determination is made in step S13, the process returns to step S11 again. On the other hand, when a condition for making an assessment is satisfied, the process proceeds to step S14.

In step S14, the response state assessment unit 313 identifies the response state of the customer 50 by making an assessment based on the customer's biological information acquired in step S11. In addition, the response state assessment unit 313 outputs the identified customer's biological information to the notification unit 314.

In step S15, the notification unit 314 generates notification information in accordance with the response state identified in step S14. Then, to give notice of the notification information to the customer 50 and the like, the notification unit 314 transmits the notification information to the display device 40. The display device 40 gives notice by displaying the notification information.

With the above-described response state assessment process, it is possible to conduct an analysis on a user who has actually encountered a commercial product or the like. For example, the customer 50 is able to study a response state, which is subjective information of the customer 50, from an objective viewpoint by consulting the notification information provided in step S15. Therefore, the customer 50 is able to evaluate a provided commercial product or the like in accordance with an index that is a customer's own response state. In addition, a business operator that sells a provided commercial product or the like obtains the response state of the customer 50 as an index of a response and reaction of the customer 50 to the provided commercial product or the like by consulting the notification information provided in step S15.

The present invention is not limited to the above-described embodiment. The present invention encompasses modifications, improvements, and the like within the scope in which an object of the present invention is achieved. The devices included in the above-described embodiment may be implemented by a general electronic device having an information processing function.

The above-described series of processes may be executed by hardware or may be executed by software. One functional block may be made up of hardware alone or may be made up of software alone or may be made up of a combination of hardware and software. In other words, the functional configuration illustrated in FIG. 2 is only illustrative and not limited. In other words, it may be enough for the assessment system S to include the function capable of executing the above-described series of processes as a whole, and which functional block is used to implement the function is not limited to the example of FIG. 2.

For example, the functional configuration included in the present embodiment may be implemented by a processor that executes arithmetic processing, and processors usable in the present embodiment include not only those made up of various processing units alone, such as a single processor, multiprocessors, and a multi-core processor, but also combinations of these various processing units and a processing circuit, such as an ASIC (application specific integrated circuit) and an FPGA (field-programmable gate array).

When a series of processes is executed by software, a program that makes up the software is installed from a network or a storage medium onto a computer or the like. A computer may be a computer incorporated in dedicated hardware. A computer may be a computer that is capable of executing various functions by installing various programs and may be, for example, a general-purpose personal computer.

A storage medium containing such a program may be provided to a user by being distributed separately from a device body to provide the program to the user or may be provided to a user in a state of being preinstalled in a device body. A storage medium that is distributed separately from a device body is made up of, for example, a magnetic disk (including a floppy disk), an optical disk, a magneto-optical disk, or the like. An optical disk is made up of, for example, CD-ROM (Compact Disc-Read Only Memory), DVD (Digital Versatile Disc), Blu-ray (registered trademark) Disc (Blue-ray Disc), or the like. A magneto-optical disk is made up of MD (Mini Disc) or the like. A storage medium that is provided to a user in a state of being preinstalled in a device body is made up of, for example, the ROM 32 of FIG. 2, in which a program is stored, a hard disk included in the storage unit 35 of FIG. 2, or the like.

In the specification, a step of describing a program to be stored in a storage medium includes, of course, a process that is executed in time series may be not always processed in time series in accordance with the order and may also include a process that is executed in parallel or executed individually. In the specification, the term “system” is defined as the meaning of an overall apparatus made up of multiple devices, multiple units, and the like.

Some embodiments of the present invention are described above; however, these embodiments are only illustrative and do not limit the technical scope of the present invention. The present invention may be implemented in other various embodiments, and, in addition, various modifications including omissions, replacements, or the like are possible without departing from the gist of the present invention. These embodiments and their modifications are included in the scope and gist of the invention described in the specification and the like and are also included in the invention described in the claims and equivalents thereof. For example, the embodiments of the present invention may be modified into the following modifications.

### First Modification

In the above-described method of assessing a response state by using the response state assessment unit 313, an assessment further using β waves in brain waves may be made. β waves are frequency components that lie at frequencies higher than α waves, and many β waves indicate that the activity of brain is active. Many β waves are generated, for example, under high stress, in an excited state, or the like. When comfort identified in the assessment according to the above-described embodiment is low and β waves are active, it is found as a state of feeling stress. In contrast, when comfort obtained in the assessment according to the above-described embodiment is high and β waves are active, it is found as a state, such as a highly concentrated state and a highly interested state. In this way, β waves are added and then an assessment is made, with the result that a response state is identified as not only a comfort and relaxed state but also a high work efficient state or the like.

### Second Modification

In the above-described assessment made by the response state assessment unit 313, an assessment may be made by using not only the customer's biological information but also environmental information. For example, the response state assessment unit 313 may identify the relationship between an environment and a response state by comparing the customer's biological information with environmental information in time series.

Specifically, such a relationship that a first response state is obtained in an environment outside a model house and a second response state is obtained in an environment after entering the model house may be identified. Thus, after entering the model house, such a causal relationship that the response state has changed can be notified as notification information. Similarly, when the operation mode of a domestic appliance, such as an air conditioner, is controlled, such a causal relationship that the response state has changed as a result of a change in the operation mode can be notified as notification information. Thus, it is possible to determine, for example, an operation mode in which the customer 50 is relaxed, an operation mode in which the customer 50 is able to concentrate on studying, and the like.

### Third Modification

In a method of making an assessment on a response state by using the above-described response state assessment unit 313, an assessment using further another biological information may be made. An assessment using, for example, any one or any combination of a number of basic body movements, a number of minute body movements, and an eye movement direction of the customer 50, which are pieces of biological information, may be made.

In this case, the biological information measurement device 10 measures the biological information of the customer 50 by using, in addition to the above-described brain wave sensor, Doppler sensor, and electrocardiographic sensor, for example, any one or any combination of a line-of-sight sensor, an image capturing apparatus, an acceleration sensor, and the like. The biological information measurement device 10 detects, for example, feature points located around eyes, such as the inner corners of the eyes, the tails of the eyes, and pupils, by using the line-of-sight sensor. The biological information measurement device 10 performs a measurement in accordance with variations in the line of sight of the customer 50 based on a change in the relative positional relationship among these detected feature points. The biological information measurement device 10 measures, for example, variations in the eye movement direction.

Other than the above, the biological information measurement device 10, for example, captures the still image or motion picture of the customer 50 by using an image capturing apparatus, such as a camera. The biological information measurement device 10 also detects the acceleration of the customer 50 with an acceleration sensor worn by the customer 50. Then, the biological information measurement device 10 measures variations in the motion of the customer 50 by making an analysis with a technique, such as a known image analysis, on information measured by any one or any combination of these image capturing apparatus, acceleration sensor, and the like. The biological information measurement device 10 measures, for example, a number of basic body movements and body movement intervals due to the motion or the like of the customer 50, a number of minute body movements and body movement intervals due to shivering or the like of the customer 50, or the like.

As in the case of the above-described embodiment, the biological information measurement device 10 generates the customer's biological information by associating these measured variations such as variations in eye movement direction and variations in the number of body movements and the like with measurement time in time series. The biological information measurement device 10 transmits the generated customer's biological information to the assessment device 30. The customer biological information acquiring unit 311 of the assessment device 30 acquires the customer's biological information by receiving the customer's biological information from the biological information measurement device 10. The customer biological information acquiring unit 311 outputs the acquired customer's biological information to the response state assessment unit 313 and the database updating unit 315.

When the response state assessment unit 313 identifies a response state as described in the above embodiment, the response state assessment unit 313 may also use the customer's biological information acquired in the present modification to identify a response state. For example, the response state assessment unit 313 identifies various motions of the customer 50 in accordance with the customer's biological information such as variations in the eye movement direction, the number of body movements, and the like, acquired in the present modification. The response state assessment unit 313 identifies, for example, the motions of the customer 50, such as minute shivering, frequent movements of hands, so-called unconscious foot and leg shaking, rubbing the eyes, nodding, and moving the line of sight to a specific direction. The response state assessment unit 313 uses these identified motions to identify a response state. For example, specific motions that are made when people tell a lie depend on individuals; however, telling a lie is mostly accompanied by some motions. The response state assessment unit 313 identifies a response state additionally in consideration of the presence or absence or the like of these identified motions.

According to the present modification thus configured, more pieces of biological information are measured and used, so it is possible to further highly accurately identify the response state of the customer 50. In addition, according to the present modification, by giving notice of these identified motions to the customer 50 as one grounds identifying a response state, so it is possible to further increase persuasion to the customer 50 in identifying the response state.

### EXPLANATION OF REFERENCE CHARACTERS

10 biological information measurement device

20 environmental information measurement device

30 assessment device

31 CPU

31 ROM

33 RAM

34 communication unit

35 storage unit

36 input unit

37 display unit

40 display device

50 customer

311 customer biological information acquiring unit

312 environmental information acquiring unit

313 response state assessment unit

314 notification unit

315 database updating unit

351 assessment information database

S assessment system

1. An assessment system comprising:
a measurement unit that measures biological information of a customer who has been presented with at least any one of a provided commercial product and a provided service;
an assessment unit that identifies a response state of the customer who has been provided with the presentation, by making an assessment based on the biological information measured by the measurement unit; and
a notification unit that gives notice based on the response state of the customer, identified by the assessment unit.

2. The assessment system according to claim 1, wherein the notification unit provides the notice to the customer for use in promoting a sale of at least any one of the provided commercial product and the provided service.

3. The assessment system according to claim 1, wherein the measurement unit further measures environmental information indicating characteristics of an environment in which the customer has been provided with the presentation, and
the assessment unit identifies a relationship between the environment and the response state by comparing the biological information of the customer, measured by the measurement unit, with the environmental information indicating the characteristics of the environment, measured by the measurement unit, in time series.

4. The assessment system according to claim 3, wherein the measurement unit measures the environmental information by using any one or any combination of a temperature sensor, a humidity sensor, an air quality sensor, an illuminance sensor, an atmospheric pressure sensor, and an acceleration sensor.

5. The assessment system according to claim 1, wherein the measurement unit measures biological information of the customer not only after the customer has been provided with the presentation but also before the customer is provided with the presentation, and
the assessment unit assesses a change in a response state of the customer based on both the biological information of the customer before the customer is provided with the presentation and the biological information of the customer after the customer has been provided with the presentation.

6. The assessment system according to claim 1, wherein the assessment unit consults a database storing multiple pieces of biological information measured in multiple environments and identifies a response state of the customer in accordance with a comparison between the multiple pieces of biological information stored in the database and biological information of the customer.

7. The assessment system according to claim 6, wherein a category based on a degree of influence of biological information on a response state is stored in the database, and
the assessment unit identifies a response state of the customer in accordance with the multiple pieces of biological information stored in the database, the category, and biological information of the customer.

8. The assessment system according to claim 6, wherein the database contains biological information measured in a place where a mind of a person of which biological information is measured is easy to be uplifted and biological information measured in a place where a mind of a person of which biological information is measured is easy to be settled as the multiple pieces of biological information.

9. The assessment system according to claim 1, wherein the measurement unit measures the biological information by using any one or any combination of a brain wave sensor, a Doppler sensor, and an electrocardiographic sensor.

10. The assessment system according to claim 1, wherein the measurement unit measures the biological information by using any one or any combination of a brain wave sensor, a Doppler sensor, an electrocardiographic sensor, a line-of-sight sensor, an image capturing apparatus, and an acceleration sensor.

11. The assessment system according to claim 1, wherein the measurement unit measures information on a motion of the customer by using a Doppler sensor, and
the assessment unit acquires a variation in a heart rate of the customer, which is biological information, in accordance with the information on the motion of the customer.

12. The assessment system according to claim 1, wherein the measurement unit measures information on a motion of the customer by using a Doppler sensor, and
the assessment unit acquires any one or any combination of a rate of respiration and a depth of respiration of the customer, which are pieces of biological information, in accordance with the information on the motion of the customer.

13. The assessment system according to claim 1, wherein the measurement unit measures information on a motion of the customer, and
the assessment unit acquires any one or any combination of a number of basic body movements, a number of minute body movements, and an eye movement direction of the customer, which are pieces of biological information, in accordance with the information on the motion of the customer.

14. An assessment method that is executed by an assessment system, the assessment method comprising:
a measuring process in which a measurement unit measures biological information of a customer who has been presented with at least any one of a provided commercial product and a provided service;
an assessment process in which an assessment unit identifies a response state of the customer who has been provided with the presentation, by making an assessment based on the biological information measured by the measurement unit; and
a notification process in which a notification unit gives notice based on the response state of the customer, identified by the assessment unit.
